(19) United States
(12) Patent Application Publication
(10) Pub. No.: US 2005/0255058 A1
(43) Pub. Date: Nov. 17, 2005

(54) Tapioca in cosmetic preparations

(75) Inventors: Rainer Kroepke, Schenefeld (DE); Jens Neilsen, Henstedt-Ulzburg (DE); Boris Syskowski, Hamburg (DE); Wiebke Lindemann, Hamburg (DE); Andreas Schaefer, Hamburg (DE); Silke Heinecke, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/085,820

(22) Filed: Mar. 21, 2005

(30) Foreign Application Priority Data
Mar. 19, 2004 (DE) 10 2004 013 633.5

(51) Int. Cl. A61K 7/42; A61K 7/06; A61K 7/11

(52) U.S. Cl. 424/59; 424/70.13

(57) ABSTRACT

The present invention relates to the use of tapioca in cosmetic preparations. In particular, the invention provides a method for reducing skin shine through application of a cosmetic preparation including tapioca. Such preparation is further beneficial for preserving skin moisture.

### FIELD OF THE INVENTION

The present invention relates to the use of tapioca in cosmetic preparations.

### BACKGROUND OF THE INVENTION

The desire to appear beautiful and attractive is naturally rooted in man. Even if the beauty ideal has undergone changes over the course of time, then the desire for a flawless outward appearance has always been the aim of humans. The condition and the appearance of the skin is a significant part of a beautiful and attractive outward appearance.

In order that the skin can completely fulfil its biological functions it requires regular cleansing and care. Cleansing the skin serves to remove dirt, perspiration, and remains of dead skin particles which form an ideal breeding ground for pathogens and parasites of all types. The skin is generally cleansed using surface-active preparations (soaps, surfactants, more rarely alcoholic preparations), which are in the form of foam-forming gels or solids (soap bars) and are rinsed off again with water following application to the skin.

Skincare products, generally creams, ointments or lotions, serve mostly to moisturize and re-grease the skin. Active ingredients are often added to them which regenerate the skin and, for example, are intended to prevent and reduce its premature ageing (e.g. the appearance of wrinkles, lines).

Besides the cleansing and care of the skin, cosmetics also have an aesthetic aim. They are intended to “improve” the outer appearance of the user to correspond to the particular cultural ideas. Cosmetics thus fulfil a psychological-social function since they increase the (optical) attractiveness of the user. Falling into this sector above all else are the “decorative” cosmetics which, with the help of dyes applied to the skin, change the appearance of the user. Indirectly, however, cleansing and care products also have a positive influence since a clean, healthy skin corresponds to people's beauty ideal.

Of particular importance in this connection is the appearance of the facial skin since the face is perceived as a “projection” of the person to a particular degree by the world at large. On the facial skin in particular, besides the classic decorative cosmetics such as eyeshadow and lipstick, the cleansing and care products have a markedly aesthetic function. There is thus, for example, a whole series of preparations for the treatment and prophylaxis of bad skin and/or acne skin.

However, greasy skin and/or very shiny areas of skin, so-called T zones, are also regarded as being visually unattractive. To treat shiny areas of skin, preparations have hitherto been supplied which comprise a series of powder raw materials, i.e. particulate raw materials, such as, for example, distarch phosphate or cyclodextrins, which absorb the sebum. However, preparations of this type have the disadvantage that they remove moisture (water) from the skin and thus lead to a dry skin sensation.

### SUMMARY OF THE INVENTION

It was therefore the object of the present invention to overcome the shortcomings of the prior art and to develop preparations which on the one hand result in an effective and long-lasting reduction of the skin shine, particularly of greasy skin, without removing excessively large amounts of water from the skin, and thus lead to a (subjectively) dry skin sensation. Surprisingly, the object is achieved through:
the use of tapioca in cosmetic preparations for reducing skin shine, in particular of greasy skin;
the use of tapioca-containing cosmetic preparations with a lipid content of from 10 to 35% by weight, based on the total weight of the preparation, for reducing skin shine, in particular of greasy skin;
the use of tapioca for preserving the skin moisture in cosmetic preparations for reducing skin shine; and
the use of tapioca in cosmetic preparations with a lipid phase, whose polar to moderately polar constituents have an interfacial tension of 5-30 mN/m, for reducing skin shine.

### DETAILED DESCRIPTION OF THE PREFERRED EMBODIMENTS

Although DE 101 57 542 describes water-in-silicone oil emulsions comprising tapioca, this specification was unable to point the way to the present invention.

Tapioca (true sago, Amylum Manihot, manioc starch, mandioc starch, manioc, cassava meal or arrowroot, Brazilian, bahia-, rio- or para-A) is the official Brazilian starch (Amilo Brazil). It is the starch obtained from the root tubers of the kinds of manioc native to South America and South East Africa, particularly esculenta Crantz (Jatropha manihot L., Manihot utilissima Pohl.), Euphorbiaceae, manioc, cassava and Manihot dulcis Manihot (J. F. Gmel.) Pax var. multifida (Grah.) Pax (Fiedler, Lexikon der Hilfstoffe [Lexicon of Auxiliaries], Editio Cantor Verlag, Aulendorf, 5th edition, 2002).

Uses advantageous according to the invention are characterized in that tapioca is present in the preparation in a total amount of 0.1-25% by weight, based on the total weight of the preparation.

Uses preferred according to the invention are characterized in that tapioca is present in the preparation in a total amount of 1-10% by weight, based on the total weight of the preparation.

Preparations according to the use in accordance with the invention may be present in various forms. Thus, for example, they may be a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol.

According to the invention, preference is given to uses which are characterized in that the preparation is in the form of an emulsion.

Accordingly, the use according to the invention in cosmetic or dermatological preparations where the latter are in the form of a multiple emulsion, microemulsion, Pickering emulsion, sprayable emulsion or a foam and the like is in accordance with the invention.

Emulsions according to the invention comprise, for example, the specified fats, oils, waxes and other fatty bodies, and water and an emulsifier, as is customarily used for such a type of formulation.

The lipid phase can advantageously be chosen from the following group of substances:
mineral oils, mineral waxes;
oils, such as triglycerides of capric acid or of caprylic acid, and also natural oils, such as, for example, castor oil, macadamia oil, avocado oil or jojoba oil, dialkyl ethers, such as, for example, di-n-octyl ethers, and dialkyl carbonates, such as, for example, di-n-octyl carbonate;
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alkyl benzoates; and
silicone oils, such as dimethylpolysiloxanes, diethylpolysiloxanes, diphenylpolysiloxanes, and mixed forms thereof.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, from the group of esters of aromatic carboxylic acids and saturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of chain length from 8 to 24, in particular 12-18, carbon atoms. The fatty acid triglycerides can, for example, be chosen advantageously from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some cases, it may also be advantageous to use waxes, for example cetyl palmitate, as the sole lipid component of the oil phase.

The oil phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of cyclic or linear silicone oils, or consist entirely of such oils, although it is preferred, apart from the silicone oil or the silicone oils, to use an additional content of other oil phase components.

Cyclomethicone (octamethylcyclotetrasiloxane) is advantageously used as silicone oil to be used according to the invention. Other silicone oils, however, are also to be used advantageously for the purposes of the present invention, for example hexamethylcyclotrisiloxane, polydimethylsiloxane, poly(methylphenylsiloxane).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, 2-methyl-1,3-propanediol, glycerol and in particular one or more thickeners which can be chosen advantageously from the group consisting of silicon dioxide, aluminium silicates, polysaccharides and derivatives thereof, e.g. hyaluronic acid, xanthan gum, hydroxypropylmethylcellulose, particularly advantageously from the group of polyacrylates, preferably a polyacrylate from the group of so-called carbopols, for example carbopol grades 980, 981, 1382, 2984, 5984, in each case individually or in combination.

In particular, mixtures of the abovementioned solvents are used. In the case of alcoholic solvents, water may be a further constituent.

Emulsions according to the invention are advantageous and comprise, for example, the specified fats, oils, waxes and other fatty bodies, and water and an emulsifier, as is customarily used for such a type of formulation.

Gels according to the invention usually comprise alcohols of low carbon number, e.g. ethanol, isopropanol, 1,2-propanediol, glycerol and water or an abovementioned oil in the presence of a thickener which, in the case of oily-alcoholic gels, is preferably silicon dioxide or an aluminium silicate, and in the case of aqueous-alcoholic or alcoholic gels is preferably a polyacrylate.

In accordance with the invention, the use according to the invention is advantageously also characterized in that tapioca in the preparation reduces the stickiness of polyols if these are present therein in a total amount of 0.1-25% by weight, based on the total weight of the preparation.

Polyols advantageous according to the invention are here in particular glycerol, sorbitol, urea and panthenol.

The cosmetic and dermatological preparations according to the invention can comprise cosmetic auxiliaries as are customarily used in such preparations, e.g. preservatives, bactericides, perfumes, substances for preventing foaming, dyes, pigments which have a colouring effect, thickeners, surface-active substances, emulsifiers, softening, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

In particular, active ingredient combinations used according to the invention can also be combined with other antioxidants and/or free-radical scavengers. Such antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives (esters, ethers, peptides, lipids, nucleotides, nucleosides and salts) thereof, and sulphoximine compounds (e.g. buthionine sulphoximines, homocysteine sulphoximine, buthionine sulphones, penta-, hexa-, heptathionine sulphoximine) in very low tolerated doses (e.g. pmol to μmol/kg), and also (metal) chelating agents (e.g. α-hydroxy fatty acids, palmitic acid, phytic acid, phytin, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, ascorbic acid and derivatives thereof such as ascorbyl palmitate, tocopherols and derivatives (e.g. vitamin E acetate), vitamin A and derivatives (vitamin A palmitate), and coniferyl benzoate of benzoin resin, rutinic acid and derivatives thereof, butylhydroxytoluene, butylhydroxyanisol, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the additional antioxidant or antioxidants, it is advantageous to choose their particular concentrations from the range from 0.001-10% by weight, based on the total weight of the formulation.

If vitamin A or vitamin A derivatives, or carotenes or derivatives thereof are the additional antioxidant or antioxidants, it is advantageous to choose their particular concentrations from the range from 0.001-10% by weight, based on the total weight of the formulation.

The preparations according to the invention can advantageously comprise astringents, preference being given here to chloride salts and complexes of aluminium and/or zirconium, particularly aluminium chlorohydrate.

Preparations according to the invention can advantageously also comprise substances which absorb UV radiation in the UVB region, where the total amount of the filter substances is, for example, 0.1% by weight to 30% by weight, preferably 0.5 to 10% by weight, in particular 1.0 to 6.0% by weight, based on the total weight of the preparations, in order to provide cosmetic preparations which protect the hair and/or the skin from the entire range of ultraviolet radiation. They can also serve as sunscreen compositions for the hair.

If the preparations according to the invention comprise UVB filter substances, these may be oil-soluble or water-soluble. Oil-soluble UVB filters advantageous according to the invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate,
esters of 2-cyano-3,3-diphenylacrylic acid, preferably ethylhexyl 2-cyano-3,3-diphenylacrylate; and
diethylhexylbutamidotriazone, 2,4,6-trianilino(p-carbo-2′-ethyl-1′-hexyloxy)-1,3,5-triazine.
Advantageous water-soluble UVB filters are, for example:
   salts of 2-phenylbenzimidazole-5-sulphonic acid, such as its sodium, potassium or its triethanolammonium salt, and the sulphonic acid itself;
   sulphonic acid derivatives of benzophenones, preferably 2-hydroxy-4-methoxybenzophenone-5-sulphonic acid and its salts; and
   sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and its salts, and 1,4-di(2-oxo-10-sulpho-3-bornylidenemethyl)benzene and salts thereof (the corresponding 10-sulphato compounds, for example the corresponding sodium, potassium or triethanolammonium salt), also referred to as benzene-1,4-di(2-oxo-3-bornylidenemethyl-10-sulphonic acid.

The list of specified UVB filters which can be used in combination with the active ingredient combinations according to the invention is of course not intended to be limiting.

The invention also provides the use of a combination of the emulsions used according to the invention with at least one UVB filter as antioxidant and the use of a combination of the active ingredient combinations used according to the invention with at least one UVB filter as antioxidant in a cosmetic or dermatological preparation.

It may also be advantageous to use UVA filters which are customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione.

Advantageous UVA filters also derive from the group of triazines, thus, for example, 2,4-bis{[4-(2-ethylhexyloxy)-2-hydroxy]phenyl}-6-(4-methoxyphenyl)-1,3,5-triazine (trade name Tinosorb® S), and from the group of triazoles, such as, for example, 2,2′-methylenebis [6-2H-benzotriazol-2-yl]-4-(1,1,3,3-tetramethylbutyl)phenol) (trade name Tinosorb® M). An advantageous water-soluble UVA filter is 2′-bis(1,4-phenylene)-1H-benzimidazole-4,6-disulphonic acid sodium salt (trade name Neo Heliopan AP®).

It is possible to use the amounts used for the UVB combination.

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, and the sulphate of barium (BaSO₄).

For the purposes of the present invention, the pigments can also advantageously be used in the form of commercially available oily or aqueous predispersions. Dispersion auxiliaries and/or solubilization promoters may advantageously be added to these predispersions.

According to the invention, the pigments may advantageously be surface-treated (“coated”), the intention being, for example, to form and/or retain a hydrophilic, amphiphilic or hydrophobic character. This surface treatment can consist in providing the pigments with a thin hydrophilic and/or hydrophobic inorganic and/or organic layer by methods known per se. The various surface coatings can also comprise water for the purposes of the present invention.

Inorganic surface coatings for the purposes of the present invention can consist of aluminium oxide (Al₂O₃), aluminium hydroxide Al(OH)₃, or aluminium oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination and/or in combination with organic coating materials.

Organic surface coatings for the purposes of the present invention can consist of vegetable or animal aluminium stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

Zinc oxide particles and predispersions of zinc oxide particles suitable according to the invention are available under the following trade names from the companies listed:

| Trade name | Coating | Manufacturer |
|---|---|---|
| Z-Cote HP1 | 2% dimethicone | BASF |
| Z-Cote | / | BASF |
| ZnO NDM | 5% dimethicone | H&R |

Suitable titanium dioxide particles and predispersions of titanium dioxide particles are available under the following trade names from the companies listed:

| Trade name | Coating | Manufacturer |
|---|---|---|
| MT-100TV | Aluminium hydroxide/ | Tayca Corporation |
| | stearic acid | |
| MT-100Z | Aluminium hydroxide/ | Tayca Corporation |
| | stearic acid | |
| Eusolex T-2000 | Alumina/simethicone | Merck KgaA |
| Titanium dioxide T805 | Octyltrimethylsilane | Degussa |
| (Uvinul TiO₂) | | |

Cosmetic preparations which are a skin-cleansing composition preferably comprise at least one anionic, nonionic or amphoteric surface-active substance, or else mixtures of such substances, the active ingredient combinations used according to the invention in aqueous medium and auxiliaries as are customarily used therefor.

The surface-active substance or the mixtures of these substances may be present in the shampooing composition in a concentration between 1% by weight and 50% by weight.

These cosmetic or dermatological preparations may also be aerosols with the customary auxiliaries used therefore.

Inventive aqueous cosmetic cleansing compositions or low-water or water-free cleansing composition concentrates intended for aqueous cleaning can comprise anionic, nonionic and/or amphoteric surfactants, for example:
conventional soaps, e.g. fatty acid salts of sodium;
alkyl sulphates, alkyl ether sulphates, alkane- and alkylbenzenesulphonates;
sulphoacetates;
sulphobetaines;
sarcosinates;
amidosulphobetaines;
sulphosuccinates;
sulphosuccinic half-esters;
alkyl ether carboxylates;
protein-fatty acid condensates;
alkylbetaines and amidobetaines;
fatty acid alkanolamides; and
polyglycol ether derivatives.

Cosmetic preparations which are cosmetic cleansing preparations for the skin may be in liquid or solid form. Besides active ingredient combinations used according to the invention, they preferably comprise at least one anionic, nonionic or amphoteric surface-active substance or mixtures thereof, if desired one or more electrolytes and auxiliaries as are customarily used therefor. The surface-active substance may be present in the cleansing preparations in a concentration between 1 and 94% by weight, based on the total weight of the preparations.

Apart from the abovementioned surfactants, the compositions according to the invention comprise water and if appropriate the additives customary in cosmetics, for example perfume, thickener, dyes, deodorants, antimicrobial substances, regreasing agents, complexing and sequestering agents, pearlizing agents, plant extracts, vitamins, active ingredients and the like.

For use, the cosmetic and dermatological preparations are applied according to the invention in an adequate amount to the skin and/or the hair in the manner customary for cosmetics.

The uses according to the invention are used in particular in skin or face creams, skin or face lotions, and day or night creams or lotions.

The examples below are intended to illustrate the present invention without limiting it. Unless stated otherwise, all of the amounts, fractions and percentages are based on the weight and the total amount or on the total weight of the preparations.

### EXAMPLES

W/O emulsions containing tapioca

| Ingredient | 1 | 2 | 3 | 4 | 5 |
|---|---|---|---|---|---|
| Triglycerol diisostearate | 1.0 | 0.5 | 0.25 | 2.0 | 3.0 |
| Diglycerol dipolyhydroxystearate | 1.0 | 1.5 | 1.75 | 3.0 | 2.0 |
| Paraffin oil | 12.5 | 10.0 | 8.0 | 5.0 | 17.5 |
| Vaseline | 8.0 | 6.0 | 5.0 | 12.0 | 2.5 |
| Hydrogenated cocoglycerides | 2.0 | 1.0 | 2.5 | 5.0 | 0.25 |
| Decyl oleate | 0.5 | 0.75 | 1.0 | 2.0 | 0.25 |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Microcrystalline cellulose | 0.5 | 1.0 | — | 0.25 | 0.1 |
| Magnesium sulphate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Sodium citrate | 0.2 | 0.05 | 0.4 | 0.3 | 2.0 |
| Perfume | q.s. | q.s. | q.s. | q.s. | q.s. |
| Ethanol | 2.0 | — | 5.0 | — | — |
| Caprylic/capric triglyceride | 2.0 | 2.5 | 3.0 | 5.0 | 0.5 |
| Potassium sorbate | 0.04 | 0.15 | 0.05 | 0.03 | 0.4 |
| Benzyl alcohol | 0.3 | 0.4 | 0.25 | 0.15 | — |
| Tapioca | 1.5 | 0.3 | 0.5 | 1.0 | 5.0 |
| Water | ad 100 | ad 100 | ad 100 | ad 100 | ad 100 |

W/O emulsions containing tapioca

| Ingredient | 6 | 7 | 8 | 9 | 10 |
|---|---|---|---|---|---|
| PEG-30 dipolyhydroxystearate | — | 0.5 | 0.25 | — | 3.0 |
| Lanolin alcohol | 1.0 | 1.5 | 1.75 | 3.0 | — |
| Paraffin oil | 12.5 | 10.0 | 8.0 | 5.0 | 17.5 |
| Vaseline | 8.0 | 6.0 | 5.0 | 12.0 | 2.5 |
| Hydrogenated cocoglycerides | 2.0 | 1.0 | 2.5 | 5.0 | 0.25 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Microcrystalline cellulose | 0.5 | 1.0 | 0.75 | 0.25 | 0.1 |
| Magnesium sulphate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Perfume | q.s. | q.s. | q.s. | q.s. | q.s. |
| 1,3-Butylene glycol | 2.0 | — | 5.0 | — | — |
| Caprylic/capric triglyceride | 2.0 | 2.5 | 3.0 | 5.0 | 0.5 |
| Sodium dehydracet | — | — | 0.05 | — | — |
| Potassium sorbate | 0.3 | 0.4 | 0.25 | 0.15 | — |
| Tapioca | 0.5 | 1.5 | 2.5 | 3.5 | 8.0 |
| Talc | — | — | 0.05 | — | 0.1 |
| Water | ad 100 | ad 100 | ad 100 | ad 100 | ad 100 |

W/S emulsion containing tapioca

| Ingredient | 11 | 12 | 13 | 14 | 15 |
|---|---|---|---|---|---|
| Cetyl PEG/PPG-10/1 dimethicone | 1.0 | — | — | 3.0 | 5.0 |
| Cyclomethicone + PEG/PPG-18/18 | 10.0 | 12.5 | 25 | — | — |
| dimethicone (90:10) | | | | | |
| Cyclomethicone | 12.5 | 15 | 28.0 | 25.0 | 17.5 |
| Dimethicone | 5.0 | 13.0 | 5.0 | 12.0 | 15.0 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Sodium citrate | 1.0 | 0.1 | 0.4 | 0.9 | 2.5 |
| Perfume | q.s. | q.s. | q.s. | q.s. | q.s. |
| Potassium sorbate | 0.4 | 0.1 | 0.05 | 0.3 | 0.4 |
| Tapioca | 1.0 | 0.5 | 2.5 | 0.25 | 5.5 |
| Cetyldimethicone | 0.5 | — | 0.7 | — | — |
| Benzyl alcohol | — | — | 0.05 | — | 0.1 |
| Water | ad 100 | ad 100 | ad 100 | ad 100 | ad 100 |

W/S emulsions

| Ingredient | 16 | 17 | 18 | 19 | 20 |
|---|---|---|---|---|---|
| Cetyl PEG/PPG-10/1 dimethicone | 1.0 | — | — | 3.0 | 5.0 |
| Cyclomethicone + PEG/PPG-18/18 | 10.0 | 12.5 | 25 | — | — |
| dimethicone (90:10) | | | | | |
| Cyclomethicone | 12.5 | 15 | 28.0 | 25.0 | 17.5 |
| Dimethicone | 5.0 | 13.0 | 5.0 | 12.0 | 15.0 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Lactic acid | 0.2 | 0.1 | 0.2 | — | — |
| Sodium lactate | 0.2 | 1.0 | 0.05 | — | — |
| Perfume | q.s. | q.s. | q.s. | q.s. | q.s. |
| Tapioca | 1.0 | 3.5 | 1.5 | 2.5 | 0.1 |
| Stearyldimethicone | 0.5 | — | 0.7 | — | — |
| Dehydracetic acid | — | — | 0.05 | — | 0.1 |
| Modified starch | — | 2.5 | — | 0.15 | — |
| Water | ad 100 | ad 100 | ad 100 | ad 100 | ad 100 |

W/O emulsions containing tapioca

| Ingredient | 21 | 22 | 23 | 24 | 25 |
|---|---|---|---|---|---|
| PEG-22 dodecyl glycol copolymer | 5.0 | 1.5 | 0.25 | — | 3.0 |
| PEG-45 dodecyl glycol polymer | 1.0 | 1.5 | 1.75 | 3.0 | — |
| Paraffin oil | 12.5 | 10.0 | 8.0 | 5.0 | 17.5 |
| Isopropyl stearate | 8.0 | 6.0 | 5.0 | 12.0 | 2.5 |
| Hydrogenated coco glycerides | 2.0 | 1.0 | 2.5 | 5.0 | 0.25 |
| Evening primrose oil | 0.5 | 0.75 | 1.0 | 2.0 | 0.25 |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Tapioca | 0.5 | 1.0 | 7.5 | 0.25 | 0.1 |
| Magnesium sulphate | 0.5 | 0.6 | 0.5 | 0.7 | 1.0 |
| Glycerol | 3.0 | 5.0 | 10.0 | 15.0 | 1.5 |
| Sodium citrate | 0.2 | 0.1 | — | — | — |
| Citric acid | 0.2 | 0.1 | — | — | — |
| Perfume | q.s. | q.s. | q.s. | q.s. | q.s. |
| 1,3-Butylene glycol | 2.0 | — | 5.0 | — | — |
| Caprylic/capric triglyceride | 2.0 | 2.5 | 3.0 | 5.0 | 0.5 |
| Potassium sorbate | 0.4 | 0.15 | 0.05 | 0.3 | 0.4 |
| Benzyl alcohol | — | — | 0.05 | — | 0.1 |
| Water | ad 100 | ad 100 | ad 100 | ad 100 | ad 100 |

W/O emulsions containing tapioca

| Ingredient | 26 | 27 | 28 | 29 | 30 |
|---|---|---|---|---|---|
| Polyglyceryl-2 dipolyhydroxystearate | 3.0 | — | 0.25 | — | 3.0 |
| Polyglyceryl-3 diisostearate | 1.0 | 3.5 | 1.75 | 2.5 | — |
| PEG-40 sorbitan isostearate | — | 2.5 | 0.5 | 3.5 | 3.0 |
| Paraffin oil | 12.5 | 10.0 | 8.0 | 5.0 | 17.5 |
| Isopropyl stearate | 8.0 | 6.0 | 5.0 | 12.0 | 2.5 |
| | 2.0 | 1.0 | 2.5 | 5.0 | 0.25 |
| Isopropyl palmitate | 0.5 | 1.0 | 0.75 | 3.0 | 0.25 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Tapioca | 0.5 | 1.0 | 0.75 | 0.25 | 0.1 |
| Magnesium sulphate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.1 | 0.3 | 1.0 |
| Sodium citrate | 0.2 | 0.3 | 0.2 | 1.5 | 0.8 |
| Perfume | q.s. | q.s. | q.s. | q.s. | q.s. |
| Caprylic/capric triglyceride | 2.0 | 2.5 | 3.0 | 5.0 | 0.5 |
| Potassium sorbate | 0.24 | 0.15 | 0.05 | 0.3 | 0.4 |
| Water | ad 100 | ad 100 | ad 100 | ad 100 | ad 100 |

Silicone in water emulsion

| Ingredient | 31 | 32 | 33 | 34 | 35 |
|---|---|---|---|---|---|
| Dimethicone copolyol, caprylic/capric | 1.0 | 2.0 | 8.0 | 3.0 | 5.0 |
| triglyceride | | | | | |
| Cyclomethicone | 12.5 | 15 | 25.0 | 10.0 | 7.5 |
| Dimethicone | 5.0 | 15.0 | 5.0 | 12.0 | 15.0 |
| Mineral oil | 0.5 | 0.75 | 1.0 | 2.0 | 0.25 |
| Phenyltrimethicone | 0.5 | 1.0 | 0.75 | 3.0 | 0.25 |
| Glycerol | 5.0 | 7.5 | 10.0 | 3.0 | 1.0 |
| Xanthan gum | — | 0.1 | — | 0.25 | 1.0 |
| Panthenol | 0.5 | 1.0 | 0.75 | 0.25 | 0.1 |
| Perfume | q.s. | q.s. | q.s. | q.s. | q.s. |
| Methylparaben | 0.4 | 0.1 | 0.05 | 0.3 | 0.4 |
| Propylparaben | 0.3 | 0.4 | 0.25 | 0.15 | — |
| Iodopropynyl butylcarbamate | — | — | 0.05 | — | 0.1 |
| Tapioca | 0.5 | 2.5 | 1.0 | 0.75 | 3.5 |
| Water | ad 100 | ad 100 | ad 100 | ad 100 | ad 100 |

O/W emulsion containing tapioca

| Ingredient | 36 | 37 | 38 | 39 | 40 |
|---|---|---|---|---|---|
| Glyceryl sterate | 1.0 | — | — | 0.5 | 0.25 |
| Polyethylene glycol(40) stearate | 10.0 | — | 5 | — | — |
| Triglycerol methylglucose distearate | — | 5.5 | — | — | 2.5 |
| Sorbitan stearate | — | 1.5 | 3 | — | — |
| Cyclomethicone | 2.5 | 15 | 8.0 | 5.0 | 7.5 |
| Dimethicone | 5.0 | 3.0 | 5.0 | 2.0 | 5.0 |
| Behenyl alcohol | 1 | — | 2 | 1 | — |
| Stearyl alcohol | — | 1 | — | 1 | — |
| Tapioca | 5 | 10 | 15 | 20 | 2.5 |
| Cetylstearyl alcohol | — | — | 1 | 1 | — |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Perfume | q.s. | q.s. | q.s. | q.s. | q.s. |
| Methylparaben | 0.4 | 0.1 | 0.05 | 0.3 | 0.4 |
| Propylparaben | 0.3 | 0.4 | 0.25 | 0.15 | — |
| Iodopropynyl butylcarbamate | — | — | 0.05 | — | 0.1 |
| Glycerol | 5 | 10 | 3 | 15 | 7.5 |
| Modified starch | — | 2.5 | — | 0.15 | — |
| Water | ad 100 | ad 100 | ad 100 | ad 100 | ad 100 |

O/W emulsion containing tapioca

| Ingredient | 41 | 42 | 43 | 44 | 45 |
|---|---|---|---|---|---|
| Polyethylene glycol(21) stearylether | 1 | — | 2.5 | 2 | 1.5 |
| Polyethylene glycol(2) stearylether | 1 | — | 5.5 | 3 | 7.5 |
| Cetearyl glucoside | — | 8 | — | — | — |
| Behenyl alcohol | 3 | 2 | — | 1 | — |
| Stearyl alcohol | 3 | 2 | — | 2 | — |
| Cetylstearyl alcohol | 3 | 4 | | — | 2 |
| Hydrogenated polyisobutene | 0.5 | 0.75 | 1.0 | 2.0 | 0.25 |
| Tapioca | 5 | 3.5 | 2.5 | 1.0 | 7.5 |
| Octyldodecanol | 0.5 | 1.0 | 0.75 | 3.0 | 0.25 |
| Glycerol | 5 | 10 | 15 | 3 | 7.5 |
| Panthenol | 0.5 | 1.0 | 0.75 | 0.25 | 0.1 |
| Perfume | q.s. | q.s. | q.s. | q.s. | q.s. |
| Methylparaben | 0.4 | 0.1 | 0.05 | 0.3 | 0.4 |
| Propylparaben | 0.3 | 0.4 | 0.25 | 0.15 | — |
| Iodopropynyl butylcarbamate | — | — | 0.05 | — | 0.1 |
| Modified starch | 0.5 | — | — | 0.15 | — |
| Water | ad 100 | ad 100 | ad 100 | ad 100 | ad 100 |

O/W emulsion containing tapioca

| Ingredient | 46 | 47 | 48 | 49 | 50 |
|---|---|---|---|---|---|
| Glyceryl stearate citrate | 1.0 | 0.5 | 0.1 | 0.5 | 0.3 |
| Polyethylene glycol(20) cetearyl ether | 10.0 | 1.0 | 5 | — | — |
| Triglycerol methylglucose distearate | — | — | — | — | 2.5 |
| Ethylbutyl acetylaminopropionate | 5 | — | 15 | 20 | — |
| Tapioca | 1.0 | 2.5 | 3.5 | 0.75 | 2.25 |
| Dimethicone | 0.5 | 3.0 | 0.75 | 1.5 | 0.2 |
| Behenyl alcohol | 1 | — | 2 | 1 | 0.2 |
| Dicaprylyl carbonate | 3 | 5 | 10 | 15 | 5 |
| Stearyl alcohol | — | — | — | 1 | 0.2 |
| Cetylstearyl alcohol | — | — | 1 | 1 | 0.2 |
| Tocopherol | 0.5 | 0.5 | 0.75 | 0.25 | 0.1 |
| Octyldodecanol | 0.5 | — | 0.75 | 3.0 | 0.25 |
| Panthenol | 0.5 | — | 0.75 | 0.25 | 0.1 |
| Carbomer | 0.05 | 0.35 | 0.15 | 0.1 | — |
| Perfume | q.s. | q.s. | q.s. | q.s. | q.s. |
| Caprylic/capric triglyceride | 1 | 5 | 3 | 5 | 10 |
| Methylparaben | 0.4 | 0.3 | 0.05 | 0.3 | 0.4 |
| Propylparaben | 0.3 | — | 0.25 | 0.15 | — |
| Iodopropynyl butylcarbamate | — | — | 0.05 | — | 0.1 |
| Phenoxyethanol | — | 0.5 | — | 0.15 | — |
| Sorbitol | 10 | — | — | 5 | — |
| Butylene glycol | — | — | — | 5 | 10 |
| Propylene glycol | — | — | 10 | 5 | — |
| Glycerol | — | 7.5 | — | — | — |
| Water | ad 100 | ad 100 | ad 100 | ad 100 | ad 100 |

O/W emulsion containing tapioca

| Ingredient | 51 | 52 | 53 | 54 | 55 |
|---|---|---|---|---|---|
| Glyceryl stearate | 2.6 | — | 2.6 | 0.5 | 0.3 |
| Caprylic/capric triglyceride | — | 0.6 | — | — | 1.5 |
| Cetyl alcohol | — | 2.5 | — | — | 2.0 |
| Glyceryl stearate citrate | — | 2.0 | — | — | 3.0 |
| Cetylstearyl alcohol | 1.5 | — | 2.5 | — | — |
| Stearyl alcohol | — | 2.5 | — | 1.25 | — |
| Polyethylene glycol(40) stearate | 0.8 | — | 0.8 | — | 2.5 |
| C12-15 alkylbenzoate | 3.0 | — | 3.0 | 2.0 | — |
| Tapioca | 3.0 | 3.0 | 3.5 | 0.75 | 2.25 |
| Beeswax | 1.0 | 3.0 | 1.0 | 1.5 | 0.2 |
| Dicaprylyl ether | — | 3.0 | — | — | — |
| Octyltriazone | 1.5 | — | 2 | 1 | 0.2 |
| BHT | 0.05 | 0.05 | 0.05 | 15 | 5 |
| Hydrogenated vegetable oil | 1.5 | 3.0 | 1.5 | 1 | 0.2 |
| Hydrogenated cocoglyceride | — | 1.5 | — | — | 0.5 |
| Tridecyl stearate + | — | 1.2 | — | — | 2.5 |
| tridecyl trimellitate + | | | | | |
| dipentaerythrityl | | | | | |
| hexacaprylate/hexacaprate | | | | | |
| Benzophenone-3 | — | — | 3.5 | — | — |
| Ethylhexyl cinnamate | — | — | 5.0 | — | — |
| Cocoglycerides | 3.0 | — | 3.0 | 1 | 0.5 |
| Phenoxyethanol, methyl-, ethyl-, butyl-, | 1.0 | 1.0 | 1.0 | 0.75 | 1.25 |
| isobutyl-, propylparaben | | | | | |
| EDTA, Na salt | — | 1.0 | — | — | 1.0 |
| Glycerol | 10.0 | 11.6 | 10.0 | 3.0 | 12.9 |
| Disodium | 1.0 | — | — | 1.5 | 2.5 |
| phenyldibenzimidazolesulphonate | | | | | |
| Carbomer, sodium salt | 0.1 | 0.2 | 0.1 | 0.1 | 0.3 |
| Perfume | q.s. | q.s. | q.s. | q.s. | q.s. |
| Iodopropynyl butylcarbamate | 0.18 | — | 0.18 | — | — |
| Cyclomethicone | 4.0 | 3.0 | 4.0 | — | 3.5 |
| Tocopherol acetate | 0.5 | 0.5 | 0.5 | 0.15 | — |
| Carrageenan | 0.1 | — | 0.1 | — | 0.1 |
| Phenyl benzimidazolesulphonic acid, | 1.0 | — | 2.0 | 0.15 | — |
| Na salt | | | | | |
| Creatine | 1.0 | 1.0 | 1.0 | 1.5 | 3.0 |
| Creatinine | 0.1 | 0.1 | 0.1 | 0.15 | 1.0 |
| Sodium ascorbyl phosphate | 0.05 | 0.05 | 0.05 | 5 | — |
| Ethylhexylglycerol | — | 0.5 | — | — | 0.35 |
| Panthenol | 1.4 | 1.4 | 1.4 | — | — |
| Retinyl palmitate | — | 0.2 | — | 0.5 | — |
| Methylpropanediol | 2.0 | — | 2.0 | — | 5.0 |
| Ethylhexyl cocoate | 2.0 | — | 2.0 | — | 4.0 |
| Coenzyme Q10 | 0.01 | 0.01 | 0.01 | — | 0.1 |
| Water | ad 100 | ad 100 | ad 100 | ad 100 | ad 100 |

1. A method for reducing skin shine comprising applying a cosmetic or dermatological preparation to the skin, wherein the preparation includes tapioca.

2. The method of claim 1, wherein the preparation further comprises a lipid content of 10-35% by weight, based on the total weight of the preparation.

3. The method of claim 1, wherein the preparation comprises a lipid phase including polar to moderately polar constituents that have an interfacial tension of 5-30 mN/m.

4. The method of claim 1, wherein the preparation preserves skin moisture.

5. The method of claim 1, wherein the preparation is in the form of an emulsion.

6. The method of claim 1, wherein the tapioca is present in the preparation in a total amount of 0.1-25% by weight, based on the total weight of the preparation.

7. The method of claim 1, wherein the tapioca is present in the preparation in a total amount of 1-10% by weight, based on the total weight of the preparation.

8. The method of claim 1, further comprising one or more polyols in a total amount of 0.1-25% by weight, based on the total weight of the preparation.

9. The method of claim 8, wherein the one or more polyol is selected from the group consisting of glycerol, sorbitol, urea, and panthenol.

10. A cosmetic or dermatological preparation for reducing skin shine, comprising tapioca and a lipid content of 10-35% by weight, based on the total weight of the preparation.

11. The preparation of claim 10, wherein the preparation comprises a lipid phase including polar to moderately polar constituents that have an interfacial tension of 5-30 mN/m.

12. The preparation of claim 10, wherein the preparation is in the form of an emulsion.

13. The preparation of claim 10, wherein the tapioca is present in the preparation in a total amount of 0.1-25% by weight, based on the total weight of the preparation.

14. The preparation of claim 10, wherein the tapioca is present in the preparation in a total amount of 1-10% by weight, based on the total weight of the preparation.

15. The preparation of claim 10, further comprising one or more polyols in a total amount of 0.1-25% by weight, based on the total weight of the preparation.

16. The preparation of claim 15, wherein the one or more polyols is selected from the group consisting of glycerol, sorbitol, urea, and panthenol.

17. A cosmetic or dermatological preparation for reducing skin shine, comprising tapioca and a lipid phase, said lipid phase including polar to moderately polar constituents that have an interfacial tension of 5-30 mN/m.

18. The preparation of claim 17, wherein the preparation is in the form of an emulsion.

19. The preparation of claim 17, wherein the tapioca is present in the preparation in a total amount of 0.1-25% by weight, based on the total weight of the preparation.

20. The preparation of claim 17, wherein the tapioca is present in the preparation in a total amount of 1-10% by weight, based on the total weight of the preparation.

21. The preparation of claim 17, further comprising one or more polyols in a total amount of 0.1-25% by weight, based on the total weight of the preparation.

22. The preparation of claim 21, wherein the one or more polyols is selected from the group consisting of glycerol, sorbitol, urea, and panthenol.
